(19) United States
(12) Patent Application Publication
(10) Pub. No.: US 2016/0317397 A1
(43) Pub. Date: Nov. 3, 2016

(54) Cleaning Composition with Dissolvable Packaging

(71) Applicant: WISYS TECHNOLOGY FOUNDATION, INC., Madison, WI (US)

(72) Inventors: Eric J. Wenz, Hudson, WI (US); An T. Trieu, Woodbury, MN (US); Andrew J. Lind, River Falls, WI (US)

(21) Appl. No.: 15/144,350

(22) Filed: May 2, 2016

Related U.S. Application Data
(60) Provisional application No. 62/155,676, filed on May 1, 2015.

(51) Int. Cl. A61K 8/02; A61K 8/46; A61Q 5/02; A61K 8/81; A61K 8/44; A61Q 19/10; A61K 8/60; A61K 8/92

(52) U.S. Cl. CPC A61K 8/0204; A61K 8/60; A61K 8/466; A61K 8/922; A61K 8/8129; A61K 8/44; A61Q 19/10; A61Q 5/02; A61K 2800/87

(57) ABSTRACT

A cleaning composition contained within a dissolvable package is provided. The package is formed at least partially of a water-soluble film material and the cleaning composition therein, which can be an organic composition, is formed without water to avoid degrading the package. In use, the package is contacted with water to dissolve the package and expose the cleaning composition contained therein.

### CROSS-REFERENCE TO RELATED APPLICATIONS

This application claims priority from U.S. Provisional Patent Application Ser. No. 62/155,676 filed on May 1, 2015, the entirety of which is expressly incorporated by reference herein.

### FIELD OF THE INVENTION

The present invention relates generally to compounds for cleaning the body and/or hair of an individual and more specifically to compounds for cleaning an individual having dissolvable packaging.

### BACKGROUND OF THE INVENTION

Many types of cleaning compositions are currently utilized to wash the body and hair of individuals. The cleansing or cleaning compositions come in a wide variety of formulations to provide the required cleaning of the individual along with other desired attributes for the cleaning composition, such as moisturizing or fragrancing, among others.

When utilizing the cleaning compositions, the compositions are contained in packaging that is suitable for the environment in which the cleaning composition is to be utilized. Most often this packaging takes the form of a plastic bottle, as the packaging needs to be able to withstand moisture to prevent the cleaning composition from coming into contact with water prior to use. Also, the plastic material forming the bottle is inert and does not chemically interact with the cleaning composition.

However, while plastic bottles are highly useful for this purpose, once the cleaning composition has been used from the bottle in its entirety, the problem arises concerning the disposal of the bottle. In certain situations, the bottle can be recycled for re-use, but many times the nature of the components of the cleaning composition make the bottle unsuitable for recycling. Further, those bottles that are not recycled often still contain a residue of the cleaning composition which can be detrimental to the environment and/or wildlife that the bottle comes into contact with.

To attempt to address this issue, other types of packaging materials have been attempted to be used with cleaning compositions of these types. However, none have been able to provide a satisfactory replacement to the plastic bottle as evidenced by the continued widespread use of these types of bottles as containers for cleaning solutions and compositions.

Therefore, it is desirable to develop a cleaning composition and accompanying packaging material to address the issue of the packaging for these cleaning compositions.

### SUMMARY OF THE INVENTION

According to one aspect of an exemplary embodiment of the invention, a cleaning composition is provided that can be packaged in a package or container formed of a dissolvable material. The dissolvable material encloses the cleaning composition in a manner that enables the cleaning composition to be isolated from the environment in which the container is placed until it is desired to use the composition. When it is desired to use the cleaning composition, the container can be contacted with or otherwise subjected to a readily available substance or material that quickly and effectively dissolves the package or container to render the cleaning composition therein available for use.

According to another aspect of an exemplary embodiment of the invention, the cleaning composition is formulated in a manner that effectively limits any interaction of the cleaning composition with the dissolvable material forming the package. By forming the cleaning composition in this manner, the package readily remains intact around the cleaning composition until such time as an individual intends to dissolve the package by contacting the package with a suitable amount of a substance or material capable of dissolving the package.

These and other aspects, features and advantages of the invention will be made apparent from the following detailed description taken together with the drawing figures.

### BRIEF DESCRIPTION OF THE DRAWINGS

The drawings illustrate the best mode currently contemplated of practicing the present invention.

In the drawings:

FIG. 1 is an isometric view showing one exemplary embodiment of a cleaning composition in the dissolvable packaging of the invention.

FIG. 2 is an isometric view showing the dissolvable packaging of FIG. 1 being dissolved to expose the cleaning composition through contact with a suitable dissolving substance.

FIG. 3 is an isometric view showing another exemplary embodiment of the package according to the invention.

FIG. 4 is a schematic view showing still another exemplary embodiment of the package according to the invention.

### DETAILED DESCRIPTION OF THE INVENTION

Before the present compositions, apparatuses and methods are described, it is understood that this invention is not limited to the particular embodiments and methodology, as these may vary. It is also to be understood that the terminology used herein is for the purpose of describing particular exemplary embodiments only, and is not intended to limit the scope of the present invention which will be limited only by the appended claims.

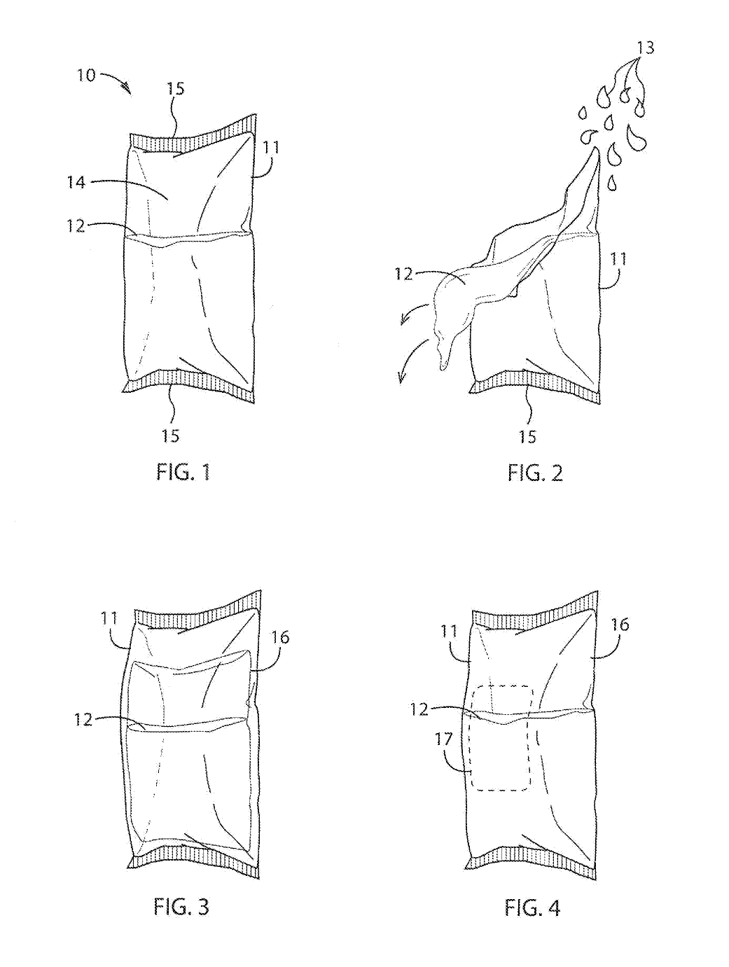

Referring now to FIGS. 1 and 2, a package for a cleaning composition formed according to one exemplary embodiment of the present invention is shown at 10. In the illustrated embodiment, the package 10 is formed with a film layer 11 of a film that can be dissolved by contacting the film with an amount of a substance that chemically reacts with the film material to break down the film material. The package 10 can be formed in any suitable manner, such as by employing sonic welding or heat sealing the layer 11 at one or more ends 15 of the package to close the package 10 or by using any suitable type of mechanical fastener (not shown) to close or seal the package 10.

In one exemplary embodiment, the material forming the package 10 is polyvinyl alcohol (PVOH or PVA), though other suitable dissolvable films and/or materials can be utilized as well such as poly(lactic-co-glycolic acid) (PLGA), Polyvinylpyrrolidone (PVP), Polyethylene glycol (PEG) and carboxymethyl cellulose. PVA is readily soluble in water, such that when the package 10 comes into contact with water 13, the water 13 will interact with the PVA material layer 11 of the package 10, breaking down or dissolving the layer 11 forming the package 10 thereby exposing the amount of the cleaning composition 12 contained within an interior 14 defined by the film layer 11, as best shown in FIG. 2.

To enable the film layer 11 to be readily dissolvable to expose the cleaning composition 12, in one exemplary embodiment the layer 11 is formed of a film of a dissolvable material, e.g., PVA, that is substantially thick enough to avoid or prevent any significant degradation by the cleaning composition 12 in order to retain the cleaning composition 12 within the interior 14 of the package 10 until it is desired to dissolve the film layer 11 and expose the composition 12. In another exemplary embodiment, to accomplish this, the film layer 11 is at least 20 μm in thickness such that incidental and/or inadvertent contact of the film layer 11 with water 13 will not completely dissolve the material forming the film layer 11. In still another exemplary embodiment, the film layer 11 is between about 20 μm about 100 μm in thickness, but also can be between about 30 μm and about 75 μm in thickness or about 50 μm in thickness.

Further, while the packaging 10 can be formed with a single layer 11 of the film material, it is also contemplated that the packaging 10 can be formed with multiple layers of the same or different films, some or all of which can be dissolvable, and some of which may not be dissolvable in order to form a barrier layer for the packaging 10 until use of the cleaning composition 12 within the packaging 10 is desired. For, example, in FIG. 3 a package 10 is formed with an inner layer 16 that is formed of a conventional packaging material, such as a polyethylene or polypropylene, where the inner layer 16 is not water soluble. Thus when the package 10 is contacted with water 13, the layer 11 dissolves to expose the layer 16, which can be formed as a partial enclosure around the cleaning composition 12 or as a complete enclosure that can be opened in a conventional manner to expose the composition 12. Alternatively, the layer 16 can be positioned around the layer 11, such that after removing the layer 16 in a conventional manner, such as by cutting or tearing the layer 16, the layer 11 can be exposed in order to contact the layer 11 with water 13.

In addition, referring to FIG. 4, in another exemplary embodiment the package 10 can be formed with portions of the package 10 formed of the respective film layers 11 and 16, such as by forming a dispensing window or opening 17 of layer 11 within layer 16. In this exemplary embodiment, when water 13 contacts the window 17, the layer 11 forming the window 17 dissolves allowing the composition to be dispensed from within the package 10 formed by the layer 16.

In order to minimize any premature interaction with the layer 11 forming all or a portion of the package 10, the cleaning composition 12 can have any of a variety of formulations, and in one exemplary embodiment includes suitable amounts of various combinations of one or more carriers, one or more surfactants, one or more thickeners, one or more fragrances and other optional ingredients, including pigments/colorants, emollients, moisturizers and vitamins, among others. Examples of these components utilized in the cleaning composition 12 include, but are not limited to: cocobetaine, coconut oil, aloe vera, grapeseed oil, xanthan gum, Panthenol B5, vegetable glycerin, citric acid, and tea tree essential oil. In one exemplary embodiment, the components of the composition 12 are each plant-based, optionally with zero free water in the composition 12.

In one exemplary embodiment of the cleaning composition 12, the composition 12 is formed with only a small amount of water, or completely without water, to eliminate any interaction of water in the composition 12 with the film material forming the packaging 10.

The following are summaries of various sample formulations of the cleaning composition 12, optionally in concentrated forms, and the tests run on them to determine the efficacy of the cleaning composition 12 and the ability of the composition 12 to be reliably held within the packaging 10 by the layer 11 until desired to dispense the composition 12 from the packaging 10. Further, in the exemplary embodiments discussed below, the cleaning composition 12 is formulated to be a shampoo, but compositions for other cleaning purposes are also contemplated as being able to form the composition 12, such as a conditioner, body wash, hand soap, shaving gel/cream, camp soap, toothpaste, stain remover, dish or fabric detergent, and/or a combination of said products.

### EXPERIMENTAL

### Formulation 1

### Mild Conditioning Shampoo, Efficacy Test with Olive Oil, Crayon, and Pva Film Testing

### Materials:

### Shampoo:

Phase A:

distilled water

Polyquaternium-10 (Lot Number 26404)

Phase B:

Alkyl Sulfonate (Sodium Laureth Sulfate)

Polyglucose:

Grapeseed oil

Phase C:

Paraben-DU (diazolidinyl urea):

Fragrance: Tea Tree Essential Oil, Lemongrass Essential Oil, Sweet Basil Essential Oil

### Packaging Film:

PVA film (Vivos®) obtained from MonoSol, of Portage, Ind.)

### Methods:

Phase A: Add polyquaternium-10 powder to the water in a mixing bowl
Then add and stir gently each ingredient from phase B one by one to phase A mixture
Add phase C to phase A/B mixture and stir again

### Observations:

### Phase A:

distilled water: clear colorless liquid

Polyquaternium-10: (conditioner) fine white powder

### Phase B:

Alkyl Sulfonate (Sodium Laureth Sulfate): (cleansing agent), clear colorless liquid

Polyglucose: (cleansing agent) dense drippy clear colorless liquid

Grapeseed oil: (emollient) slightly yellow clear colorless liquid

### Phase C:

Paraben-DU: thick clear colorless liquid

Fragrance: tea tree oil and basil oil have distinct fragrance smell

Trial 1 (Making the Shampoo with 1/10 Scale of Ingredients):

Observations
The polyquaternium-10 was added in a mixing bowl that contained the weighed water. It slowly dissolved turning into gel-like consistency in 5 minutes.
Phase B was added into phase A after phase A had turned into gel. The solution was mixed, and it turned to a cloudy “egg white” consistency. Some clear pockets observed were determined to be from the phase A gel.
Phase C was then added into the mixture. The same consistency was observed. Further mixing created soapy bubbles on the surface of the mixture.
Final shampoo mixture was runny and liquid in consistency, having the slight fragrance of tea tree oil, and cloudy and white in color.

Efficacy Testings
Test with Olive Oil: 7 drops of shampoo mixture were added to a fabric square that had been treated with a few droplets of oil on palm. The testing area was agitated/rubbed for approximately 1 minute, resulting in the formation of fine white bubbles. The fabric square was then rinsed with water and let dry with paper towels. The oil spot was not observed indicating that the shampoo formulation successfully cleaned the olive oil stain.
Test with crayon: Crayon was soiled on another fabric square. The same procedure was repeated as with olive oil. Following cleaning with shampoo formulation, no crayon was observed on the fabric test once again indicating that the shampoo formulation was successful in cleaning the crayon stain.

### Trial 2

Observations

In manufacture of shampoo instead of letting the phase A settle, phase B ingredients were added directly into bowl containing phase A and stirred in between. Basil oil was used as a fragrance additive. Both ingredients from phase C were added, and a final mixing was performed to fully incorporate the ingredients. All ingredients were uniformly dissolved, and no clear pockets were seen as observed during trial 1.

Efficacy Tests

The efficacy tests with the olive oil and the crayon were repeated as for trial 1. The shampoo completely cleaned the fabric tested.

### Trial 3: Waterless Shampoo Formulation

### Observations

Alkyl sulfonate was added to the polyquaternium-10 without addition of water. Upon mixing, the mixture formed white clumps apart from the clear slightly gelatinous liquid of the alkyl sulfonate.
Polyglucose was added, and clumping was observed. However, upon addition of grapeseed oil, the mixture turned to a milky white gel.
After adding the paraben and approximately 6 drops of lemongrass oil and mixing, the mixture started to demonstrate a white, pasty appearance. Further mixing resulted in a sticky paste that was white and fluffy in consistency.

### Efficacy Tests (Repeated as for Trials 1 and 2)

Oil containing fabric square was successfully cleaned using shampoo formulation from trial 3.
Some crayon residues remained, indicating that this formulation did not clean the crayon stain as well as that in trials 1 or 2.

### Trial 4: 10% WATER Shampoo Formulation in a 1/10 Scale

### Observations

The shampoo mixture mixed well and was less pasty than the waterless formulation in trial 3.
A pH of 8 was observed.

### Efficacy Tests (Repeated as for Trials 1-3)

Oil and crayon were not observed on fabric squares following cleaning with shampoo formulation of trial 4, indicating that such formulation successfully cleaned both stains.

TABLE 1
SHAMPOO FORMULATIONS
1/10 scale of all ingredients from original formula

| | | Actual | Actual | Actual | Actual |
|---|---|---|---|---|---|
| | | mass | mass | mass | mass |
| | Mass in | TRIAL | TRIAL | TRIAL | TRIAL 4 |
| | Theory | 1 | 2 | 3 | (10% water) |
| DI water | 14.16 g | 14.2 g | 14.1 g | 0 g | 1.098 g |
| Poly- | 0.12 g | 0.12 g | 0.120 g | 0.120 g | 0.121 g |
| quaternium- | | | | | |
| 10 | | | | | |
| Alkyl | 7.2 g | 7.16 g | 7.19 g | 7.21 g | 7.202 g |
| Sulfonate | | | | | |
| Polyglucose | 1.9 g | 1.90 g | 1.89 g | 1.96 g | 1.929 g |
| Grapeseed | 0.24 g | 0.24 g | 0.25 g | 0.247 g | 0.297 g |
| Oil | | | | | |
| Paraben-DU | 0.24 g | 0.24 g | 0.28 g | 0.255 g | 0.257 g |
| Fragrance | 0.12 g | 0.12 g | 0.14 g | 0.13 g | 0.128 g |

### PVA Film Testing:

1. PVA film was cut into 3×3 squares
2. Testing with water: the film dissolved quickly in water.
3. Testing with the shampoo formulations:
The film was placed on a weighing boat, and a few drops of the freshly made shampoo (trial 1) were placed on film. The film started to curl up and was partially dissolved once it came in contact with the shampoo. After a few minutes of incubation, a deep socket was observed on the film surface.
Another piece of film was stretched on both sides using metal clamps with rubber ends. A tensile strength test was performed using a small metal ball (mass of 3.535 g) that was close to expected mass of the cleaning composition/SoPod. A few drops of shampoo (trial 2) were placed on the stretched film, and the ball was carefully placed on it. It took 9:56 minutes for the ball to break through with pressure.
   For Trial 3 formulation, the ball and shampoo on a stretched PVA film stayed intact, and no breakage was observed with the film.
   For Trial 4 formulation, the film remained intact for a short amount of time however broke after handling suggesting that the water contributed to the degradation of the film.

### Formulation 2

### Full Batch of the Waterless Formulation (Trial 3), Packaging, and Home Testings

### 1. Observations During Mixing:

Significant clumping was observed as a result of there being no water to dissolve the polyquaternium-10 in the formulation after addition of each ingredient in phase B.
Further mixing turned the mixture to a fluffy “whipped egg white” consistency.
By first mixing a more diluted and liquid ingredient like grapeseed oil with the polyquaternium-10, less clumping was observed.
   2. Formulated shampoo was injected into films. Select samples were sealed with an Impulse Sealer PFS-200 (PP/PE Bags, AC 110V), while others were left out in open air, and the remaining samples were housed in a closed glass jar.

### 3. At Home Testing:

At Home Trial #1

Once SoPOD capsule (measuring approximately 4 cm×2 cm, weighing approximately 2 g and containing the waterless formulation) came into contact with 102 degree F. water, the pod was observed to hold its shape for approximately 10 seconds, however, during this time the pod was dissolving slowly. Following approximately 5 minutes of agitation by hand to break up the pod, a thick white later formed. Resulting shampoo was used to clean and rinse hair without formation of any residue.

At Home Trial #2

The shampoo turned into a clear gel within the pod. As a result the amount was insufficient to clean the hair.

At Home Trial #3

Same results observed from trial 2 after 12 hours.

### Formulation 3

### Modifications of the Inorganic Shampoo from Formulation 2 into Organic Shampoo

All packages (trial 1 and 2) that were left out in open air turned slightly yellow in color. The shampoo within turned into clear thick gel and lost its integrity. Some clear clumps remained visible. The shampoo samples that were housed in the closed jar lost their volume with the shampoo condensing into a thick white gel which formed at the bottom of the jar with bubbles observed on top.

### Materials:

½ cup of coco betaine
½ cup of aloe vera
¼ cup of coconut oil/jojoba oil/argan oil
1 TBS of glycerin
1 tsp of xanthan gum
Essential oil (fragrance)

### Procedure:

1. Each of the above ingredients were blended in a blender with the exception of xanthan gum and the essential oil;
2. Following blending, xanthan gum was added until desired thickness was reached;
3. Essential oil was added to desired fragrance;
   Adjustments to formulation were made if the shampoo was too moisturizing by addition of a higher concentration of aloe vera and addition of higher concentrations of oil if the shampoo was too dry.
   Increased concentration of coco betaine can be utilized to increase cleaning properties.

Unit Conversions:

½ cup=4 oz=113.398 g, ¼ cup=56.699 g

ITBS=1.5 oz=42.5243 g

1 tps=0.1667 oz=4.726 g

TABLE 2
ORGANIC WATERLESS SHAMPOO FORMULATIONS

| | Mass in | Actual mass |
|---|---|---|
| | theory | in TRIAL 5 |
| coco betaine | 11.34 g | 11.24 g |
| aloe vera | 11.34 g | 11.40 g |
| coconut oil | 5.670 g | 5.674 g |
| glycerin | 4.252 g | 4.675 g |
| xanthan gum | 0.4126 g | 0.410 g |
| essential oil | a few drops | 7 drops of tea tree oil |
| Grapeseed oil | | 34.016 g |

### Observations:

coco betaine: clear runny gel-like liquid

aloe vera: clear runny gel-like liquid

coconut oil: white solid, melted easily into a clear liquid when being heated

glycerin: clear thick gel-like

xanthan gum: very fine white powder

essential oil

All ingredients were easily mixed into a milky shiny white cream.

### Efficacy Testing (Cleaning and Film Degradation):

Fabric squares tested by repeating procedure used on Trials 1-4 were clean of oil and crayon, indicating that shampoo formulation was successful in cleaning oil and crayon stain.
The film stayed intact when packaged.

Home testing revealed shampoo formulation to consist of a milky gel with some lather, however, samples left in open air dehydrated and residue remained after testing. Cleansing with formulation left hair and skin dry and dehydrated.

### Formulation 4

### Variations on Formulation 3 by Making Shampoo Using Lower Concentrations of Aloe Vera and Higher Concentrations of Oil, and Making Shampoo Using Olive Oil, with Further Observations on the Organic Shampoo

1. 50% Aloe Vera (compared to the original (trial 3) formulation
¼ cup of the aloe vera was used instead of 2 cup.
½ cup of coconut oil was used instead of V cup
   An increased concentration of tea tree oil was also utilized.
   The above formulation resulted in a nice and similar consistency.
Sample pods stored in a closed jar remained soft, and shampoo formulation remained consistent and resulted in optimal lathering and bubbles. When tested, hair and hands felt less dry. Further modification could be made in order to increase smoothing and moisturizing effects by lowering aloe vera or coco betaine content.

2. Formulation without Aloe Vera

With no aloe vera, the shampoo formulation was observed to lose its consistency and was not tested further.

3. Formulating the half aloe vera formulation with olive oil
¼ cup of aloe vera
¼ cup of olive oil (Addition of olive oil resulted in a clear yellowish green oil that turned the shampoo to a yellowish white color)

All other ingredients remained the same

4. Humidity Testing—Materials and Methodology

For humidity testing a XIKAR PuroTemp Digital Hygrometer with Wireless Remote 837XI-2 was utilized, 70 F (temperature) and 92% (humidity), along with a desiccator, with tap water poured in and kept at bottom of the desiccator.
2 shampoo bags and the hygrometer were placed on a piece of metal drainer, with a closed cap sealing the open edges with greasy wax.
The test samples were incubated over 48 hours and were observed to remain intact during this time.
The test samples were rinsed with water, and it was noted that the package took a longer time between agitations to dissolve.

5. Pressure Testing
Weight was applied to the sample packages and they were observed to remain intact. When further pressure was applied by hand, the package burst. Further pressure testing should be conducted to ascertain limits of package.
Testing conducted on the Olive Oil shampoo package demonstrated the formulation and packaged reacted well to water. Minimal clumping was observed however there was a loss of shampoo while trying to dissolve the film, however it was noted that there was sufficient shampoo remaining to utilize for cleansing. No lathering was observed, however, hair and skin was left soft and clean.

### Formulation 5

### Making Shampoo Using Grapeseed Oil, Research on Moisturizing Ingredients, Packaging with 30 Micron Film

1. Making Shampoo Using Grapeseed Oil:
Actual mass used: recorded in the data table 2 above
Observations: Shampoo had increased liquidity, therefore an increased concentration of xanthan gum was added in order to increase thickening.

2. Extra Ingredients Ordered:
extra coconut oil, coco betaine for future usage
hydrolyzed wheat protein and panthenol since these are the ingredients used in conditioner

3. Testing of 30 Micron Film:
The 30 micro film was observed to be significantly thinner than previous film used.
The shampoo formulation softened and made the package appear wrinkled however the package remained intact.
One left in closed package, one left in open air for next day observation.
Observation: the film got weakened fast once touched and squeezed slightly.

### Pressure and Humidity Testing on the 30 Micron Film, Experimentation on Tighter Packaging

1. Pressure Test:
Weight was applied to the sample package, however, uniform weight distribution was not possible. Further advanced testing on pressure parameters is needed.

2. Humidity Test:
Packages were incubated in the presence of 78% Humidity, at 72 F for 1 hour (mimicking the maximum time the package may remain in the shower/bathing environment): the package completely ruptured.

3. Packaging:
In order to make the package more aesthetically appealing, the amount of air inside the packaging was increased by switching to a plastic pipette instead of a syringe. It was demonstrated that 76 micron film held the shampoo better than the 30 micron film.
Optimal method for packaging: the pack should be slightly longer than desired with an open end and a closed end, the shampoo should be injected up to half if the length of the pack, optionally in conjunction with air. The package should be sealed at the very top end of the pack, while folding down the film to force all remaining air to the bottom of the pack, and repeat hot seal for tighter pack, such as in a vertical fill/form/seal (VFFS) machine.

TABLE 3
OTHER EXEMPLARY WATERLESS SHAMPOO/CLEANING
COMPOSITION FORMULATIONS

| Ingredients | Mass (g) | Mass (%) | | |
|---|---|---|---|---|
| coco betaine | 11.324 g | 33.01% | 5.61 | polyglucose 5.43 |
| aloe vera | 5.68 g | 16.56% | 6.86 | cocoglucose 2.14 |
| coconut oil | 5.681 g | 16.56% | 5.44 | propylene glycol 16.58 |
| glycerin | 4.3 g | 12.54% | 24.34 | apricot oil 4.93 |
| xanthan gum | 0.511 g | 1.49% | | |
| essential oil | 0.5 g | 1.46% | | |
| panthenol B5 | 0.507 g | 1.48% | 0.57 | |
| grapeseed oil | 5.675 g | 16.55% | 8.45 | |
| citric acid | 0.12 g | 0.35% | | |

TABLE 4
INGREDIENT LIST AND PROPERTIES

| Ingredient | Manufacturer | Description/Properties |
|---|---|---|
| propylene | essential depot | hygroscopic, |
| glycol | USP- Kosher food | clear liquid |
| | grade 1 quart | |
| coco betaine | makingcosmetics | (Amphosol CG, found at |
| | 500 ml/16.8 floz | Voyageur): Amphoteric. A |
| | | humectant. Good foaming |
| | | properties. Good flash foam and |
| | | foam stabilization properties. |
| | | Adding this amphoteric to an |
| | | anionic mix will reduce the |
| | | harshness of the other surfactants |
| | | and changes the viscosity. It is a |
| | | good anti-static for hair. Poor to |
| | | mild cleanser. Good where |
| | | mildness is vital—babies or |
| | | sensitive skin—but primarily |
| | | used as a seconday surfactant. |
| | | mild amphoteric surfactant |
| | | derived from coconut oil, |
| | | highly water soluble in |
| | | wide pH range, pH 5-6, |
| | | 30% active substance |
| | | clear yellow liquid |
| grapeseed oil | now solutions | light, odorless |
| | 473 ml/16.8 floz | liquid |
| | | mild astringent and |
| | | emollient properties |
| | | contain essential fatty |
| | | acids: linoleic, oleic, |
| | | stearic, palmitic, myristic, |
| | | lauric acids. |
| | | soothe dryness and |
| | | irritation |
| aloe vera oil | Dr. Adorable Inc | |
| | 455 g/16 oz | |
| avocado oil | now solutions | support skin elasticity, |
| | | nutrient rich, hydrating |
| polyglucose | makingcosmetics | mild non-ionic surfactant |
| | 250 ml/8.4 floz | composed of sugar |
| | | (glucose) dervied from |
| | | coconuts, foam stabilizer, |
| | | low de-fatting but good |
| | | wetting properties, improve |
| | | conditioning effects of |
| | | cationic surfactants, soluble |
| | | in water, pH 11-12, 50% |
| | | active substances |
| | | clear golden liquid, |
| | | viscous, no odor |
| xanthan gum | now real food | thickening agent |
| | 170 g/6 oz | |
| sweet almond | now solutions | nourishing, promotes |
| oil | | healthy skin, |
| coco glucose | makingcosmetics | mild surfactant blend, |
| | 250 ml/8.4 floz | clear yellow liquid, low |
| | lot number: | viscosity with characteristic |
| | | odor |
| vegetable | now solutions | derived from non-GMO |
| glycerin | 473 ml/16 floz | palm oil or vegetable oil, |
| | | softening, lubricating, skin |
| | | cleanser |
| coconut oil | viva labs | |
| | 473 ml/16 floz | |
| | lot: 15146 | |
| citric acid | ball | |
| | 212 g/7.5 oz | |
| apricot oil | now solutions | |

(All raw materials have been tested and all remained stable in the film)

TABLE 5
ADDITIONAL WATERLESS SHAMPOO FORMULATIONS

| | | | Predicted/ | | | | |
|---|---|---|---|---|---|---|---|
| | | | typical use | | | | |
| | Mass (g) | Mass (%) | surfactants: | 50 g | 100 g | 100 g | |
| Ingredients | Before | Before | 15-40% | trial 1 | trial 2 | trial 3 | 500 g |
| coco betaine | 11.324 | 33.01 | 30% | 12.5 | 25 | 30.09 | 149.45 |
| coco glucose | | | 5% | 2.5 | 5 | 5.52 | 25.75 |
| polyglucose | | | 5% | 2.5 | 5 | 6.56 | 25.78 |
| propylene | | | 5% | | 8.36 | 5.01 | 25.38 |
| glycol | | | | | | | |
| aloe vera | 5.68 | 16.56 | 10% | 8 | 6.16 | 9.27 | 45.36 |
| hydrolyzed | | | 1% | 0.5 | 1.11 | 1.03 | 6.2 |
| wheat protein | | | | | | | |
| hydrolyzed | | | 1% | 0.5 | 1.2 | 1.03 | 6.08 |
| keratin protein | | | | | | | |
| glycerin | 4.3 | 12.54 | 12% | 6 | 7.95 | 12.54 | 59.93 |
| panthenol B5 | 0.507 | 1.48 | 2% | 0.5 | 1.21 | 1.99 | 9.73 |
| grapeseed oil | 5.675 | 16.55 | 8% | 6 | 2.20 | 8.2 | 41.24 |
| apricot oil | | | 8% | 2 | 2.02 | 8.40 | 41.69 |
| tea tree oil | | | 2% | 1 | | | |
| peppermint oil | | | | | | | |
| lemon oil | | | | | | | |
| (fragrance) | | | | | | | |
| almond oil | | | 2% | | | 4.93 | 9.37 |
| coconut oil | 5.681 | 16.56 | 2% | 2 | 2.1 | 2.01 | 16.21 |
| essential oil | 0.5 | 1.46 | | | | | |
| citric acid | 0.12 | 0.35 | 0.5% | | | | |
| xanthan gum | 0.511 | 1.49 | | put in too | added | added | added |
| | | | | much | less | lesser | the |
| | | | | | than | than | least |
| | | | | | before | before | amount |
| DI water | | | | | | | a few |
| | | | | | | | drops |
| | | | | pH 5.3 | pH 4.77 | pH 5.3 | pH ~5 |
| | | gel-up | | too thick | | | still a |
| | | phase | | and | | | bit |
| | | separated | | drying | | | drying |

(With the new shampoo formulations, all ingredients were added one after another. Liquid surfactants and oils don't mix as well, using hand-held blender (for baking type) solved the phase-separating problem. New formula has an opaque white color and has a citrus, tea tree, and peppermint scent to it).

The following are listings of those components found to be compatible with the PVA film and therefore capable of use in the cleaning composition of exemplary embodiments of the invention.

List of PVA-Compatible Surfactants for Use in Formulations:

Cetearyl Alcohol

Cetyl Alcohol

Cocamidopropyl Hydroxysultaine

Decyl Glucoside

Disodium Cocoamphodiacetate

Disodium Cocoyl Glutamate

Peg 7 Glyceryl Cocoate

Potassium C12-14 Alkyl Phosphate

Sodium C14-16 Olefin Sulfonate

Sodium Cocoyl Alaninate

Sodium Cocoyl Glutamate

Sodium Cocoyl Isethionate

Sodium Lauroyl Methyl Isethionate

Sodium Methyl Cocoyl Taurate

Ingredient Declaration List

List of PVA-Compatible Carrier Oils

Apricot oil

Sweet almond oil

Grape seed oil

Avocado oil

Olive oil

Sesame oil

Evening primrose

Canola (Rapeseed)

Soybean oil

Sunflower oil

Jojoba oil

Emu oil

Castor oil

Borage seed oil

Walnut oil

Peanut oil

Pecan oil

Macadamia oil

Fractionated coconut oil

Hazelnut oil

Coconut oil

List of PVA-Compatible Essential Oils

Allspice

Aniseed

Basil

Bay

Benzoin

Bergamot

Black pepper

Cajuput

Calamus

Camomile

Camphor

Caraway

Carrot seed

Cassia

Cedarwood

Chamomile

Cinnamon

Citronella

Clary sage

Clove

Coriander

Cypress

Dill

Eucalyptus

Fennel

Frankincense

Geranium

Ginger

Grapefruit

Helichrysum

Hyssop

Jasmine

Juniper

Lavandin

Lavender

Lemon

Lemongrass

Lemon verbena

Lime

Mandarin

Marjoram

Melissa

Myrrh

Neroli

Niaouli

Nutmeg

Orange

Origanum

Palma rosa

Patchouli

Peppermint

Petitgrain

Pimento

Pine

Rose

Rose geranium

Rosemary

Rosewood

Sage

Sandalwood

Spearmint

Spikenard

Tagetes

Tangerine

Thyme

Tea tree

Vetiver

Ylang-ylang

Further in another exemplary embodiment of the invention, the cleaning composition has the following characteristics:
Water not to exceed 12%
Total dry surfactant load is at 30%
Xanthan gum is removed to resolve gelling issue.
Citric acid is removed and replaced with Phenoxyethanol and Ethylhexylglycerin as preservatives. Due to low water content, preservative level is at 0.25%

In still another exemplary embodiment of the invention, the cleaning composition has the following composition:

| | | Recommended |
|---|---|---|
| | | Load (by weight |
| Ingredient Name | Property | of the batch) |
| Cocamidopropyl Hydroxysultaine | Surfactant | 5-30% |
| Glycerine | Humectant | 1-30% |
| Olive Oil PEG-7 Esters | Emulsifier | 1-30% |
| peg 40 hydrogenated castor oil | Emulsifier | 1-30% |
| Peg 7 Glyceryl Cocoate | Surfactant/ | 1-30% |
| | Emulsifier | |
| Cocos Nucifera (Coconut) Oil | Emulsifier | 1-30% |
| DI Water | Solvent | 1-12% |
| Isostearic Acid | Emollient | 1-10% |
| Sodium C14-16 Olefin Sulfonate | Surfactant/Foam | 1-10% |
| | Booster | |
| Olive Oil Glycereth-8 Esters | Emulsifier | 1-10% |
| Panthenol | Moisturizer/B5 | 1-5% |
| | Vitamin | |
| Mentha Piperita | Fragrance | ≦3% |
| (Peppermint) Oil | | |
| Cymbopogon Flexuosus Oil | Fragrance | ≦3% |
| Melaleuca Alternifolia | Fragrance | ≦3% |
| (Tea Tree) LeafOil | | |
| Phenoxyethanol (and) | Preservative | ≦2% |
| Ethylhexylglcerin | | |
| Benzyl Alchol | Solvent | ≦1.5% |

Overall, it has been determined found that in exemplary embodiments of the cleaning composition with a low-water liquid shampoo/soap formulation, simpler is better. It has also been found that in an exemplary embodiment of the cleaning composition, heating the water/surfactant mix to no more than 200 OF during the first phase allows for better incorporation and less separation with the emulsifiers. Coconut oil should be heated to no more than 200° F. before mixing.

In addition to the above exemplary embodiments of the invention, in other exemplary embodiments the cleaning composition 12 present within the package 10 is a personal cleaning composition, such as a soap, body wash or gel, shampoo, or other suitable composition, that is for use directly on the body of an individual in order to clean the skin or hair of the individual. In those embodiments where the cleansing composition 12 is a personal cleaning composition, in certain exemplary embodiments the cleansing composition can be formed with water not to exceed 12% w/w, or in other exemplary embodiments the water can be present in an amount of up to 1% w/w, of up to 3% w/w, or up to 5% w/w, of up to 7% w/w, of up to 9% w/w or up to 11% w/w. Additionally, the surfactant load or surfactant(s) can be present in the cleaning composition 12 in amounts of up to 30% w/w, of up to 25% w/w, of up to 20% w/w, of up to 15% w/w, of up to 10% w/w or of up to 5% w/w. Further, in other exemplary embodiments, the package 10 is formed to slowly degrade when coming into contact with water, and optionally to require warm water, e.g., water with a temperature over 100° F., to effectively dissolve the package 10 and/or some mechanical action or friction applied to the package 10 in conjunction with the water, e.g., rubbing or scrubbing of the package 10, in order to effectively and completely degrade and dissolve the film forming the package 10. Additionally, the package 10 can be formed with differing thicknesses of the film for different products, such as a thinner film for a soap or more solid cleaning composition, and a thicker or multilayer film for use with a shampoo or more liquid cleaning composition. In these exemplary embodiments, these features provide certain safeguards against the premature or inadvertent dissolution of the film forming the package 10.

The following references are each expressly incorporated herein by reference in their entirety:

panthenol:

http://en.wikipedia.org/wiki/Panthenol

original organic shampoo:

http://www.natural-organic-products-and-recipes.com/howtomakeshampoo.html

conditioner recipe:

http://www.natural-organic-pnhlducts-and-recies.com/natural-conditioner.html

Vivofilm on food packaging article:

http:/www.monosol.com/vivos/MonoSol_Vivos_10229.pdf

EFSA Journal on PVA film:

http://www.efsa.europa.eu/en/sedos/doc/294.pdf

MSDS of PVA film

http://www.emsdiasum.com/microscopy/technical/msds/19800.pdf

MSDS of the Monofilm taken from Monosol website

http:/www.monosol.com/userfiles/file/MMSDS/M8630%20and%20M8630K%20Rev9.pdf

Various other embodiments of the present invention are contemplated as being within the scope of the filed claims particularly pointing out and distinctly claiming the subject matter regarded as the invention.

We claim:

1. A cleaning package comprising:
a) a film layer defining an interior and formed of a dissolvable material; and
b) an amount of the cleaning composition within the interior.

2. The cleaning package of claim 1 wherein the cleaning composition does not contain water.

3. The cleaning package of claim 1 wherein the cleaning composition comprises:
a) a carrier;
b) a surfactant; and
c) a thickener.

4. The cleaning package of claim 3 wherein the cleaning composition comprises coco betaine, aloe vera, at least one of coconut oil/jojoba oil/argan oil, glycerin and xanthan gum.

5. The cleaning package of claim 3 wherein the cleaning composition comprises coco betaine, aloe vera, olive oil, at least one of coconut oil/jojoba oil/argan oil, glycerin and xanthan gum.

6. The cleaning package of claim 3 wherein the cleaning composition comprises coco betaine, aloe vera, olive oil, coconut oil, glycerin and xanthan gum.

7. The cleaning package of claim 1 wherein the cleaning composition is a shampoo.

8. The cleaning package of claim 7 wherein the cleaning composition comprises cocamidopropyl hydroxysultaine, glycerine, olive oil PEG-7 esters, PEG 40 hydrogenated castor oil, PEG 7 glyceryl cocoate, cocos nucifera (coconut) Oil, DI water, isostearic acid, sodium C14-16 olefin sulfonate, olive oil glycereth-8 esters, panthenol, mentha piperita (peppermint) oil, cymbopogon flexuosus oil, melaleuca alternifolia (tea tree) leaf oil, phenoxyethanol (and) ethylhexylglycerin, and benzyl alcohol.

9. The cleaning package of claim 8 wherein the cleaning composition does not include water not in excess of 12% w/w.

10. The cleaning package of claim 8 wherein the cleaning composition does not include dry surfactant in excess of 30% w/w.

11. The cleaning package of claim 8 wherein the cleaning composition does not include xanthan gum.

12. The cleaning package of claim 1 further comprising:
a) a first layer formed of an insoluble material; and
b) a second layer formed of the dissolvable material.

13. The cleaning package of claim 12 wherein the first layer and the second layer are not co-extensive.

14. The cleaning package of claim 12 wherein one of the first or second layer are joined to one another to form the package.

15. The cleaning package of claim 1 wherein the dissolvable film is a polyvinyl alcohol.

16. The cleaning package of claim 1 wherein the cleaning composition is organic.

17. A method for cleaning an individual comprising the steps of:
a) providing the cleaning package of claim 1;
b) contacting the exterior layer with a material capable of dissolving the exterior layer to expose the cleaning composition therein.

18. The method of claim 17 wherein the cleaning composition comprises cocamidopropyl hydroxysultaine, glycerine, olive oil PEG-7 esters, PEG 40 hydrogenated castor oil, PEG 7 glyceryl cocoate, cocos nucifera (coconut) Oil, DI water, isostearic acid, sodium C14-16 olefin sulfonate, olive oil glycereth-8 esters, panthenol, mentha piperita (peppermint) oil, cymbopogon flexuosus oil, melaleuca alternifolia (tea tree) leaf oil, phenoxyethanol (and) ethylhexylglycerin, and benzyl alcohol.

19. A method of forming the cleaning package of claim 1 comprising the steps of:
a) forming the exterior layer into a package having a closed end and an open end;
b) injecting the cleaning composition into the package; and
c) sealing the open end of the package.
